(19) United States
(12) Patent Application Publication
(10) Pub. No.: US 2011/0202112 A1
(43) Pub. Date: Aug. 18, 2011

(54) SYSTEM AND METHOD FOR DETECTING INTERMITTENT INTERRUPTIONS IN ELECTRICAL STIMULATION THERAPY OF A PATIENT

(76) Inventor: Jonathan Ruais, McKinney, TX (US)

(21) Appl. No.: 12/858,252

(22) Filed: Aug. 17, 2010

Related U.S. Application Data
(60) Provisional application No. 61/235,272, filed on Aug. 19, 2009.

(51) Int. Cl. A61N 1/36

(52) U.S. Cl. 607/60

(57) ABSTRACT

In one embodiment, a method of identifying a cause of intermittent interruption in stimulation therapy, comprises: communicating a signal by an external controller device to an implantable pulse generator to initiate a diagnostic mode; generating a stimulation pulses by the implantable pulse generator for application to tissue of the patient through one or more electrodes of a stimulation lead during the diagnostic mode; measuring impedance values for stimulation pulses applied to tissue of the patient through the stimulation lead during the diagnostic mode; directing the patient to perform one or more physical movements while the implantable pulse generator is operating in the diagnostic mode; processing the impedance values to identify time-domain limited variations in the impedance measurements from an expected value range; and displaying on the external controller device identification of one or more electrodes exhibiting intermittent electrical breaks or shorts in accordance with the processed impedance measurements.

### CROSS-REFERENCE TO RELATED APPLICATIONS

This application claims the benefit of U.S. Provisional Application No. 61/235,272, filed Aug. 19, 2009, which is incorporated herein by reference.

### TECHNICAL FIELD

The present application is generally related to methods for identifying causes of intermittent interruptions in stimulation therapy provided by a neurostimulation system and neurostimulation systems employing the same.

### BACKGROUND

Neurostimulation systems are devices that generate electrical pulses and deliver the pulses to nerve tissue to treat a variety of disorders. Spinal cord stimulation (SCS) is the most common type of neurostimulation. In SCS, electrical pulses are delivered to nerve tissue in the spine typically for the purpose of chronic pain control. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals, which are also electrically coupled to the wire conductors, that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

### SUMMARY

In one embodiment, a method of identifying a cause of intermittent interruption in stimulation therapy, comprises: communicating a signal by an external controller device to an implantable pulse generator to initiate a diagnostic mode; generating stimulation pulses by the implantable pulse generator for application to tissue of the patient through one or more electrodes of a stimulation lead during the diagnostic mode; measuring impedance values for stimulation pulses applied to tissue of the patient through the stimulation lead during the diagnostic mode; directing the patient to perform one or more physical movements while the implantable pulse generator is operating in the diagnostic mode; processing the impedance values to identify time-domain limited variations in the impedance measurements from an expected value range; and displaying on the external controller device identification of one or more electrodes exhibiting intermittent electrical breaks or shorts in accordance with the processed impedance measurements.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF DRAWINGS

FIG. 1 depicts a stimulation system that generates electrical pulses for application to tissue of a patient.

FIG. 2 depicts a process for identifying potential causes of intermittent changes in stimulation therapy according to one representative embodiment.

FIG. 3 depicts a user interface screen for display by an external controller device according to one representative embodiment.

FIG. 4 depicts another user interface screen for display by an external controller device according to one representative embodiment.

FIG. 5 depicts a graph of impedance values that are indicative of an intermittent break according to one representative embodiment.

FIG. 6 depicts a graph of impedance values that are indicative of an intermittent short according to one representative embodiment.

### DETAILED DESCRIPTION

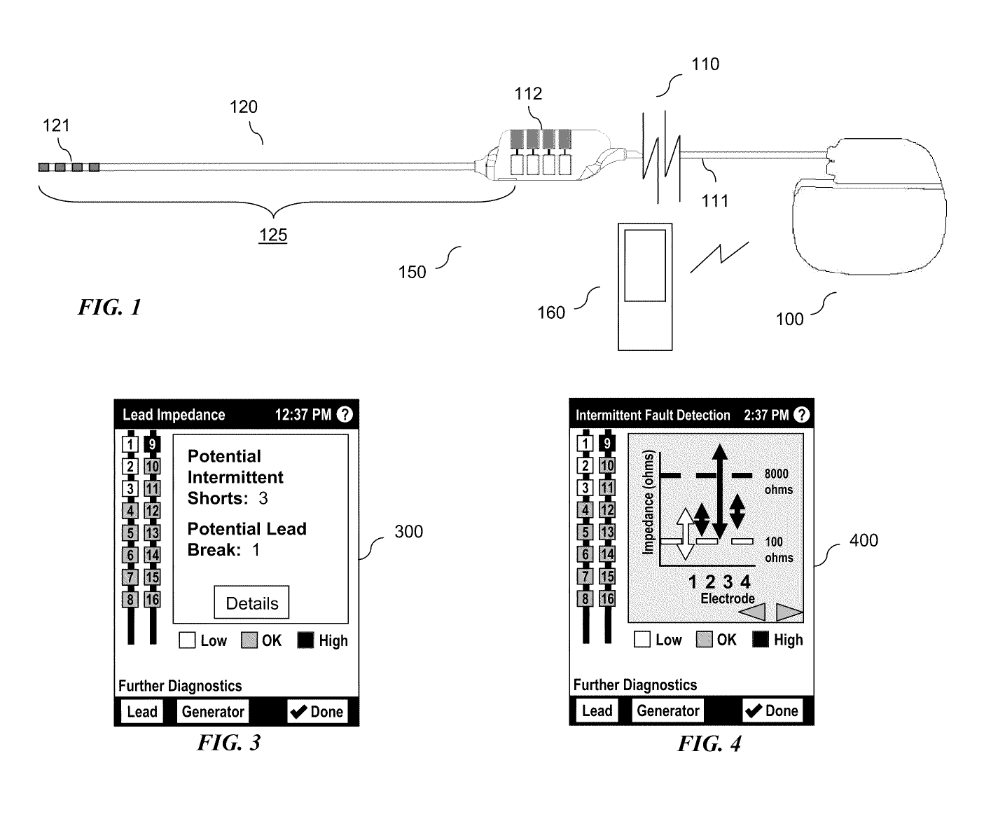

FIG. 1 depicts stimulation system 150 that generates electrical pulses for application to tissue of a patient. System 150 may be adapted to function as a spinal cord stimulation (SCS) system. System 150 may alternatively stimulate any other tissue in a patient such as peripheral nerve tissue.

System 150 includes implantable pulse generator 100 that is adapted to generate electrical pulses for application to tissue of a patient. Implantable pulse generator 100 typically comprises a metallic housing that encloses pulse generating circuitry, control circuitry, communication circuitry, battery, charging circuitry, etc. of the device. The control circuitry typically includes a microcontroller or other suitable processor for controlling the various other components of the device. Software code is typically stored in memory of the pulse generator 100 for execution by the microcontroller or processor to control the various components of the device. An example of pulse generating circuitry is described in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. A processor and associated charge control circuitry for an implantable pulse generator is described in U.S. Patent Publication No. 20060259098, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. patent Ser. No. 11/109,114, entitled “IMPLANTABLE DEVICE AND SYSTEM FOR WIRELESS COMMUNICATION,” which is incorporated herein by reference.

Stimulation system 150 further comprises stimulation lead 120. Stimulation lead 120 comprises a lead body of insulative material about a plurality of conductors that extend from a proximal end of lead 120 to its distal end. The conductors electrically couple a plurality of electrodes 121 to a plurality of terminals (not shown) of lead 120. The terminals are adapted to receive electrical pulses and the electrodes 121 are adapted to apply stimulation pulses to tissue of the patient. Also, sensing of physiological signals may occur through electrodes 121, the conductors, and the terminals. Additionally or alternatively, various sensors (not shown) may be located near the distal end of stimulation lead 120 and electrically coupled to terminals through conductors within the lead body 111.

Stimulation system 150 optionally comprises extension lead 110. Extension lead 110 is adapted to connect between pulse generator 100 and stimulation lead 120. That is, electrical pulses are generated by pulse generator 100 and provided to extension lead 110 via a plurality of terminals (not shown) on the proximal end of extension lead 110. The electrical pulses are conducted through conductors within lead body 111 to housing 112. Housing 112 includes a plurality of electrical connectors (e.g., “Bal-Seal” connectors) that are adapted to connect to the terminals of lead 120. Thereby, the pulses originating from pulse generator 100 and conducted through the conductors of lead body 111 are provided to stimulation lead 120. The pulses are then conducted through the conductors of lead 120 and applied to tissue of a patient via electrodes 121.

In practice, stimulation lead 120 is implanted within a suitable location within a patient adjacent to tissue of a patient to treat the patient's particular disorder(s). The lead body extends away from the implant site and is, eventually, tunneled underneath the skin to a secondary location. Housing 112 of extension lead 110 is coupled to the terminals of lead 120 at the secondary location and is implanted at that secondary location. Lead body 111 of extension lead 110 is tunneled to a third location for connection with pulse generator 100 (which is implanted at the third location).

Controller 160 is a device that permits the operations of pulse generator 100 to be controlled by a clinician or a patient after pulse generator 100 is implanted within a patient. Controller 160 can be implemented by utilizing a suitable handheld processor-based system that possesses wireless communication capabilities. Software is typically stored in memory of controller 160 to control the various operations of controller 160. Also, the wireless communication functionality of controller 160 can be integrated within the handheld device package or provided as a separate attachable device. The interface functionality of controller 160 is implemented using suitable software code for interacting with the clinician and using the wireless communication capabilities to conduct communications with IPG 100.

Controller 160 preferably provides one or more user interfaces that are adapted to allow a clinician to efficiently define one or more stimulation programs to treat the patient's disorder(s). Each stimulation program may include one or more sets of stimulation parameters including pulse amplitude, pulse width, pulse frequency, etc. IPG 100 modifies its internal parameters in response to the control signals from controller 160 to vary the stimulation characteristics of stimulation pulses transmitted through stimulation lead 120 to the tissue of the patient.

Conventional neurostimulation systems provide the functionality to measure the impedance associated with various electrode combinations. As currently performed, the impedance measurements only permit persistent electrical breaks and shorts to be identified. For example, if an internal wire within the lead body of the stimulation lead becomes broken, conventional neurostimulation leads are capable of detecting the high impedance associated with the break. However, if an electrical connection within the neurostimulation lead intermittently breaks or shorts, conventional stimulation systems are incapable of detecting the impedance variation. For example, as a patient moves, the patient movement may temporarily subject the stimulation lead to variable forces which disconnect a necessary electrical path or alternatively connect two otherwise independent electrical paths. After such variable forces are removed, the electrical connections may resume their previous fully functional state(s). Accordingly, the patient may subjectively perceive changes in the patient's experience of the stimulation therapy, but the cause of the patient's perception may be very difficult to identify without explanting the various components of the system and performing an intensive fault analysis of the components.

System 150 is adapted to detect the underlying cause(s) of intermittent changes in stimulation therapy experienced by a patient. In some embodiments, controller 160 causes pulse generator 100 to enter a diagnostic mode in which pulses are applied through electrode of lead(s) 120 and impedance measurements are taken to detect abrupt changes in impedance. Specifically, impedance measurements may be obtained every 0.1 seconds or less for each electrode or electrode combination under test according to one embodiment. The impedance measurements are then subjected to processing to identify potential intermittent shorts and breaks. That is, rather than time-averaging the individual impedances measurements over a lengthy test period, the individual impedance measurements are examined to identify abrupt changes in impedance values which may be indicative of electrical shorts or breaks.

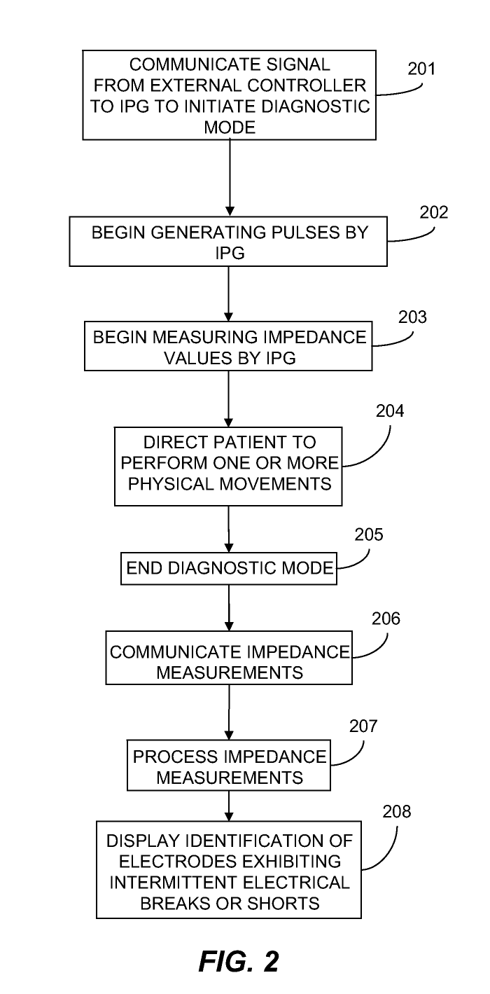

FIG. 2 depicts a process for identifying potential causes of intermittent changes in stimulation therapy according to one representative embodiment. Various portions of the process are performed by software executed by one or more of the external controller 160 and pulse generator 100. Other portions of the process are performed by hardware components of the respective devices. Some portions of the process, as discussed below, involve interaction between the patient and a respective clinician.

In 201, a signal is communicated by an external controller device to the implantable pulse generator to initiate a diagnostic mode. In 202, the implantable pulse generator begins generating pulses for application to tissue of the patient through one or more electrodes of the stimulation lead during the diagnostic mode. The stimulation pulses may be generated according to previously stored stimulation parameters defined for the patient therapy. Alternatively, the stimulation pulses may be generated by rotating the output of stimulation pulses among the various outputs of the pulse generator 100. Also, the stimulation pulses may occur at “sub-threshold” levels where the stimulation pulses do not cause a perceptible effect on the patient.

In 203, the implantable pulse generator begins measuring impedance values for stimulation pulses applied to tissue of the patient through one or more electrodes of the stimulation lead during the diagnostic mode. Preferably, the impedance measurements are made at a relatively fine time resolution. For example, impedance measurements could be obtained at every 0.1 seconds or less during the diagnostic mode for each electrode or electrode combination under test.

In 204, the clinician directs the patient to perform one or more physical movements while the implantable pulse generator is operating in the diagnostic mode. The patient movements permit the various components of the system to be subjected to various forces to bring to light an intermittent short or break in one or more electrical paths through the system.

In 205, the diagnostic mode ends, either automatically after a predetermined amount of time or by communication of an explicit command from the external controller 160 to pulse generator 100. In 206, the impedance data is communicated from the pulse generator 100 to external controller 160.

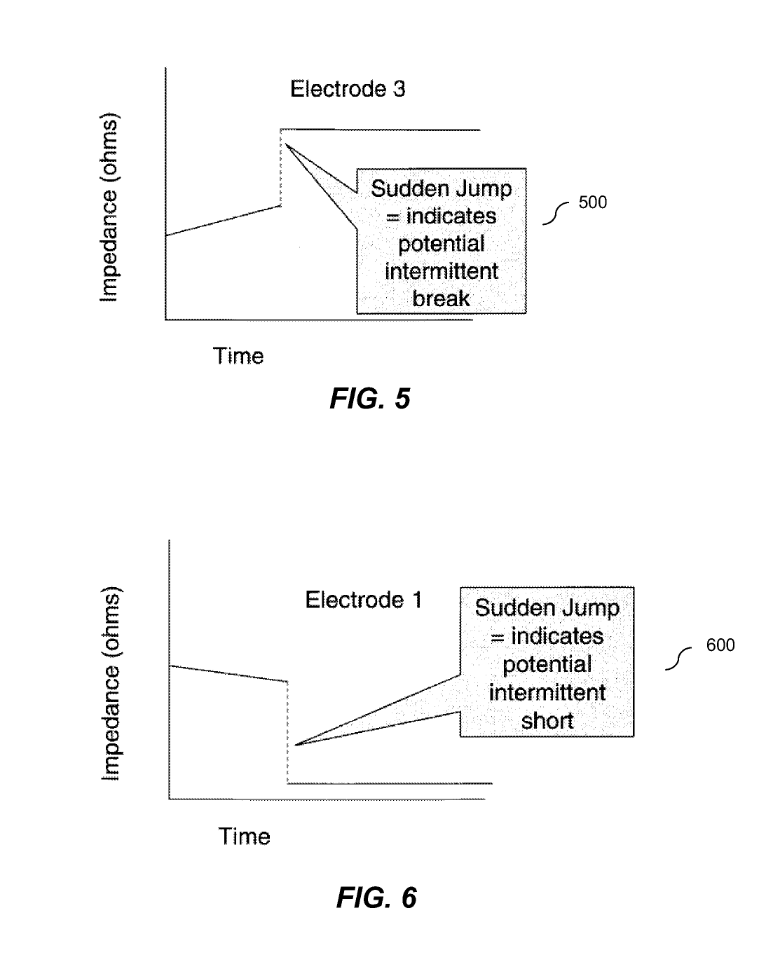

In 207, external controller 160 processes the impedance values to identify time-domain limited variations in the impedance measurements from an expected value range. In some embodiments, external controller 160 identifies individual impedance values in the data falling below 200 Ohms which are indicative of intermittent shorts. In some embodiments, external controller 160 identifies individual impedance values in the data exceeding 3000 Ohms which are indicative of intermittent breaks in the respective electrical path(s). In an alternative embodiment, the processing may occur within pulse generator 100 and the results of the processing communicated to external controller 160. Also, sudden jumps in impedance values (see graph 500 in FIG. 5) or sudden drops in impedance values (see graph 600 in FIG. 6) may be identified in the impedance data.

Referring again to FIG. 2, in 208, the external controller 160 displays identification of one or more electrodes exhibiting intermittent electrical breaks or shorts in accordance with the processed impedance measurements.

FIG. 3 depicts user interface screen 300 for display by external controller 160 according to one representative embodiment. Interface screen 300 displays the results of impedance testing for intermittent shorts and breaks. Screen 300 may graphically identify the various electrodes subjected to impedance testing. Further, screen 300 preferably graphically identifies any electrode which may exhibit an intermittent break or short. As shown in FIG. 3, electrodes 1-3 are identified as potentially having an intermittent short and electrode 9 is identified as potentially having an intermittent break. The total number of electrodes potentially having a short and/or an intermittent break may also be identified. Screen 300 includes a graphical control that permits the clinician to obtain additional data by navigating to screen 400 as shown in FIG. 4. User interface 400 provides a range of impedance values for each electrode tested during the diagnostic mode of operation of implantable pulse generator 100.

By positively detecting intermittent breaks and shorts in a neurostimulation system, more effective and more efficient decision-making can be made by a clinician according to some embodiments. That is, the clinician need not wait an inordinate amount of time to build a history of patient experience to detect intermittent breaks or shorts. Instead, the clinician is able to analyze data objectively to determine whether one or more leads should be explanted. Additionally, by identifying the specific electrode(s) involved, the entire system need not be replaced and only the affected stimulation lead need be explanted if deemed appropriate by the clinician.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

1. A method of identifying a cause of intermittent interruption in stimulation therapy provided by a neurostimulation system implanted in a patient, the stimulation system comprising an implantable pulse generator and at least one stimulation lead, comprising:
communicating a signal by an external controller device to the implantable pulse generator to initiate a diagnostic mode;
generating a plurality of stimulation pulses by the implantable pulse generator for application to tissue of the patient through one or more electrodes of the stimulation lead during the diagnostic mode;
measuring impedance values for stimulation pulses applied to tissue of the patient through one or more electrodes of the stimulation lead during the diagnostic mode;
directing the patient to perform one or more physical movements while the implantable pulse generator is operating in the diagnostic mode;
processing the impedance values to identify time-domain limited variations in the impedance measurements from an expected value range; and
displaying on the external controller device identification of one or more electrodes exhibiting intermittent electrical breaks or shorts in accordance with the processed impedance measurements.

2. The method of claim 1 wherein the processing comprises:
identifying impedance values exceeding an impedance limit value.

3. The method of claim 1 wherein the processing comprises:
identifying impedance values falling below a minimum impedance value.

4. The method of claim 1 wherein the processing comprises:
identifying abrupt changes in impedance values over a time-period of less than one second.

5. The method of claim 1 further comprising:
communicating impedance values from the implantable pulse generator to the external controller, wherein the processing of the impedance values is performed by software code executed by a processor of the external controller device.

6. The method of claim 1 further comprising:
communicating a second signal from the external controller to the implantable pulse generator to cause the implantable pulse generator to exit the diagnostic mode.

7. The method of claim 1 wherein the generating comprises:
applying stimulation pulses through outputs of the implantable pulse generator according stimulation parameters defined by one or more patient therapy programs.

8. The method of claim 1 wherein generating comprises:
applying stimulation pulses through outputs of the implantable pulse generator by rotating stimulation pulses through each output of the implantable pulse generator.

9. The method of claim 1 wherein the generating comprises:
applying stimulation pulses at amplitudes below perception threshold for the patient.

10. The method of claim 1 further comprising:
displaying a range of impedance values by the external controller for each electrode used to apply stimulation to tissue of the patient during the diagnostic mode.

11. A neurostimulation system, comprising:
an implantable pulse generator for generating stimulation pulses;
at least one implantable stimulation lead for applying stimulation pulses to tissue of a patient; and
an external controller for controlling operations of the implantable pulse generator, the external controller comprising a processor and memory storing software code, the software code comprising:
(i) first code for communicating a signal to the implantable pulse generator to cause the implantable pulse generator to enter a diagnostic mode, wherein the pulse generator generates a plurality of stimulation pulses for application to tissue of the patient through one or more electrodes of the stimulation lead and measures impedance values for stimulation pulses applied through the one or more electrodes during the diagnostic mode;
(ii) second code for receiving impedance data from the diagnostic mode from the implantable pulse generator;
(iii) third code for processing the impedance data to identify time-domain limited variations in the impedance data from an expected value range; and
(iv) fourth code for displaying identification of one or more electrodes exhibiting intermittent electrical breaks or shorts in accordance with the processed impedance measurements.

12. The system of claim 11 wherein the third code is operable to identify values in the impedance data exceeding an impedance limit value.

13. The system of claim 11 wherein the third code is operable to identify values in the impedance data falling below a minimum impedance value.

14. The system of claim 11 wherein the third code is further operable to identify abrupt changes in values in the impedance data over a time-period of less than one second.

15. The system of claim 11 wherein the implantable pulse generator, during the diagnostic mode, is operable to apply stimulation pulses through outputs of the implantable pulse generator according stimulation parameters defined by one or more patient therapy programs.

16. The system of claim 11 wherein the implantable pulse generator, during the diagnostic mode, is operable to apply stimulation pulses through outputs of the implantable pulse generator by rotating stimulation pulses through each output of the implantable pulse generator.

17. The system of claim 11 wherein the implantable pulse generator, during the diagnostic mode, is operable to apply stimulation pulses through outputs of the implantable pulse generator at amplitudes below stimulation threshold values for the patient.

18. The system of claim 11 wherein the fourth code is further operable to display a range of measured impedance values by the external controller for each electrode used to apply stimulation to tissue of the patient during the diagnostic mode.
